(19) United States
(12) Patent Application Publication
(10) Pub. No.: US 2025/0340596 A1
(43) Pub. Date: Nov. 6, 2025

(54) METHOD FOR PREPARING A SALT OF ISOCYCLOSPORIN A

(71) Applicant: DOMPE' FARMACEUTICI S.P.A., Milano (IT)

(72) Inventor: Sonia PIUMATTI, Bra (CN) (IT)

(21) Appl. No.: 18/723,078

(22) PCT Filed: Dec. 21, 2022
(86) PCT No.: PCT/IB2022/062583

§ 371 Date: Jun. 21, 2024

(30) Foreign Application Priority Data
Dec. 24, 2021 (IT) 102021000032651

(51) Int. Cl. C07K 7/64; B01J 4/00; B01J 19/00; B01J 19/24; H05B 6/80

(52) U.S. Cl. CPC C07K 7/645; B01J 4/007; B01J 19/0013; B01J 19/2445; H05B 6/802; B01J 2204/002; B01J 2219/00033; B01J 2219/00141; B01J 2219/00162

(57) ABSTRACT

The present invention belongs to the technical field of drug synthesis. In particular, the present invention is related to a for preparing a salt of isocyclosporin A, in particular by transesterification of cyclosporin A into a salt of isocyclosporin A.

### CROSS REFERENCE TO RELATED APPLICATION

This application is a 35 U.S.C. § 371 National Stage application from PCT/IB2022/062583, filed Dec. 21, 2022, which claims the benefit of Italian Application No. 102021000032651, filed Dec. 24, 2021, the disclosures of which are incorporated herein by reference.

### FIELD OF THE INVENTION

The present invention belongs to the technical field of drug synthesis. In particular, the present invention is related to a for preparing a salt of isocyclosporin A, in particular by transesterification of cyclosporin A into a salt of isocyclosporin A.

### BACKGROUND ART

Cyclosporines are oligopeptides with a cyclic structure with antifungal and immunosuppressive properties, used to modulate the body's immune response in organ transplantations, to prevent rejection.

Since the original discovery of cyclosporin, several natural cyclosporines have been isolated and identified, whereas non-natural cyclosporines have been obtained by semisynthetic methods or through the application of culture techniques. Cyclosporin A is the cyclosporin mostly used as drug.

The main indication of cyclosporin A, used in monotherapy or in association with other immunosuppressive drugs, is the prevention of rejection in organ transplantation, in particular in kidney, pancreas, liver and heart transplantations.

Cyclosporin A can also be used for the treatment of autoimmune diseases such as for example uveitis, rheumatoid arthritis, psoriasis and ulcerative colitis.

Cyclosporin has a complex chemical structure, as it is formed by 11 peptides and contains several N-methylated amino acids. As a result, the synthesis by peptide condensation reagents is rather time-consuming and complicated. Therefore, at present, the method mostly used for the synthesis of cyclosporin is by fermentation of two fungi: Trichoderma polysporum and Cylindrocarpon lucidum (Survase, S. A., Kagliwal, L. D., Annapure, U. S. & Singhal, R. S. Cyclosporin A—a review on fermentative production, downstream processing and pharmacological applications. Biotech. Advances. 29, 418-435 (2011). However, this method of synthesis does not allow a high yield of cyclosporin.

In 2010, the research group led by the chemist Danishevsky tried to synthesize cyclosporin A by condensation reaction of isonitrile in the liquid phase. However, this method of synthesis requires the use of many condensation reagents and is, therefore, complicated. At present, therefore, the solid-phase synthesis cannot be realized, except with many difficulties and over long periods of time.

Furthermore, the use of cyclosporin A is limited by its low bioavailability and high toxicity, in particular nephrotoxicity. In fact, after the oral administration of cyclosporines, the concentration level in the blood reaches a high peak, followed by a rapid decline. Consequently, the oral administration of effective quantities of cyclosporin can lead to transient but dangerously high concentrations of cyclosporin in the blood at the peak level of blood concentration, resulting in several side effects, in particular kidney and liver damages.

It has been recently observed that some isocyclosporines, in particular isocyclosporines A, B, D and G, have an improved pharmacokinetic profile with respect to cyclosporines. Advantageously, the isocyclosporines, i.e. the isomers of cyclosporines, are absorbed by the intestine in the relatively inactive and non-toxic iso-form and are subsequently converted into the pharmacologically active cyclosporin form, thus reducing peak concentrations in the blood after the administration.

The purpose of the present invention is to provide a method for preparing a salt of isocyclosporin A in order to overcome the issues encountered in cyclosporin synthesis methods present today, briefly described above.

### SUMMARY OF THE INVENTION

The Applicant has developed a method for preparing a salt of isocyclosporin A by direct conversion of cyclosporin A. The isocyclosporin obtained by the method according to the invention can be used as drug instead of cyclosporin, because the isocyclosporin has a better pharmacokinetic profile.

A first embodiment of the present invention refers to a method for preparing a salt of isocyclosporin A by transesterification of cyclosporin A into a salt of isocyclosporin A, which comprises the steps of:
a) dissolving said cyclosporin A in anhydrous methanol and adding trifluoroacetic acid;
b) heating the solution obtained according to step a) to a temperature ranging from 50° C. to the reflux temperature of the reaction mixture for a time ranging from 30 to 60 hours;
c) removing said methanol and excess of said trifluoroacetic acid;
d) recovering the salt of isocyclosporin A with said trifluoroacetic acid, wherein the molar ratio of said trifluoroacetic acid and said methanol in the solution obtained according to step a) is 1:3.

The Applicant has observed that with the optimal molar ratio between acid compound, in particular trifluoroacetic acid, and methanol of 1:3 a conversion of 53% of cyclosporin A in isocyclosporin A without having by-products is obtained, whereas with molar ratios between acid compound and methanol of 1:1 or 1:4 there is a lower conversion (30% or 20%) of cyclosporin A in isocyclosporin A (see examples 1 and 2). On the other hand, with a molar ratio between acid compound and methanol of 3:1 there is a greater conversion of cyclosporin A in isocyclosporin A (75%) but with a higher number of by-products (example 2).

In light of this, the molar ratio of 1:3 resulted to be the best ratio because the 53% of cyclosporin is converted in iso-form, without by-products, so that the unconverted residue can be recycled.

In a second embodiment of the present invention, the solution of cyclosporin A and methanol according to step a) is heated by microwave.

According to said second embodiment, the method for preparing a salt of isocyclosporin A by transesterification of cyclosporin A into a salt of isocyclosporin A comprises the steps of:
a) dissolving said cyclosporin A in anhydrous methanol and adding trifluoroacetic acid;
b) heating in a microwave oven the solution obtained according to step a);
c) removing said methanol and the excess of said trifluoroacetic acid;
d) recovering the salt of isocyclosporin A with said trifluoroacetic acid.

In particular, microwave heating according to step b) of the method is carried out at a temperature ranging from 55° C. to 65° C. for a time ranging from 10 to 20 hours, preferably of about 15 hours.

In a particularly preferred embodiment, step b) is carried out at 60° C. for 15 hours.

In fact, microwave heating at 60° C. for 15 hours allows to obtain a yield of isocyclosporin A or of a salt thereof of 100%.

A second aspect of the second embodiment of the present invention refers to a continuous flow microwave system for preparing a salt of isocyclosporin A according to the method of the present invention.

Said continuous flow microwave system comprises one or more dispensing units of starting reagents, one or more microwave reactors and one or more product collectors.

In particular, the starting reagents are supplied in one or more microwave reactors using one or more pumps, preferably one or more HPLC pumps or syringe pumps.

The system according to the invention has also one or more coolers, and one or more back pressure regulators.

In a preferred embodiment, said continuous flow microwave system comprises multiple microwave reactors in parallel.

The combination of microwave heating with the continuous flow technique advantageously allows to increase the yields of isocyclosporin A obtained.

### DETAILED DESCRIPTION OF THE INVENTION

In order to reduce the side effects due to high concentrations of cyclosporin in the blood after the oral administration, the Applicant has conceived a method for preparing a salt of isocyclosporin A, isomer of cyclosporin A, which provides for the transesterification of cyclosporin A into a salt of isocyclosporin. This method allows to overcome the problems encountered in the methods for preparing a salt of isocyclosporin A, due to its complicated chemical structure.

In fact, cyclosporin is a hydrophobic cyclical undecapeptide having the following formula I:

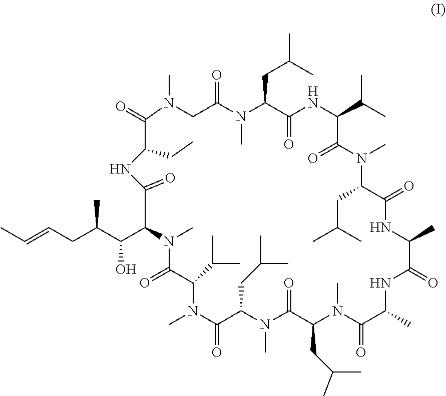 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

The isocyclosporin A, isomer of the cyclosporin A has instead the following formula (II)

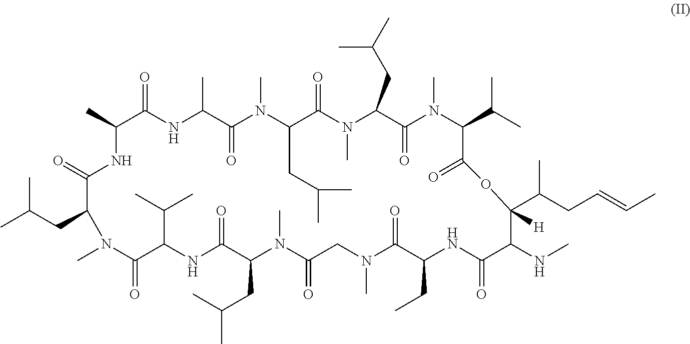 [SMILES: C/C=C/CC(C)[C@H]1OC(=O)[C@H](C(C)C)N(C)C(=O)[C@H](CC(C)C)N(C)C(=O)C(CC(C)C)N(C)C(=O)C(C)NC(=O)[C@H](C)NC(=O)[C@H](CC(C)C)N(C)C(=O)C(C(C)C)NC(=O)[C@H](CC(C)C)N(C)C(=O)CN(C)C(=O)[C@H](CC)NC(=O)C1NC]

The structural differences between the cyclosporin A and its isomer are represented in the following scheme 1:

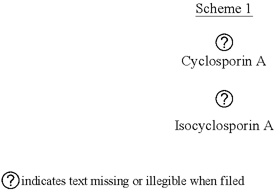

Isocyclosporin A is absorbed by the intestine in the iso-form, which is relatively inactive and non-toxic, and subsequently converted into the pharmacologically active form of cyclosporin, thus reducing peak concentrations in the blood after the administration. Therefore, isocyclosporin A can be used instead of cyclosporin A as it has the same pharmacological effects but is less toxic.

Object of the first embodiment of the present invention is a method for preparing a salt of isocyclosporin A by transesterification of cyclosporin A into a salt of isocyclosporin A, which comprises the steps of:
a) dissolving said cyclosporin A in anhydrous methanol and adding trifluoroacetic acid;
b) heating the solution obtained according to step a) to a temperature ranging from 50° C. to the reflux temperature of the reaction mixture for a time ranging from 30 to 60 hours;
c) removing said methanol and the excess of said trifluoroacetic acid;
d) recovering the salt of isocyclosporin A with said trifluoroacetic acid,
wherein the molar ratio of said trifluoroacetic acid and said methanol in the solution obtained according to step a) is 1:3.

The Applicant has advantageously observed that a molar ratio between trifluoroacetic acid and methanol in the solution formed in step a) (comprising cyclosporin A, trifluoroacetic acid and methanol) equal to 1:3 allows to obtain a yield of salt of isocyclosporin A of 80%. In an embodiment, the solution according to step a) comprises about 2 mmol of cyclosporin A and 60 mmol of methanol (see table 1 in example 1).

In particular, in the method according to the invention, step b) is carried out at a temperature ranging from 50° C. to the reflux temperature of the reaction mixture, preferably at the temperature of 60° C.

In a particularly preferred embodiment, the solution according to step a), i.e. comprising cyclosporin A dissolved in methanol, is heated for 48 hours, preferably at the temperature of 60° C.

The reaction scheme according to the method of the invention is indicated below:

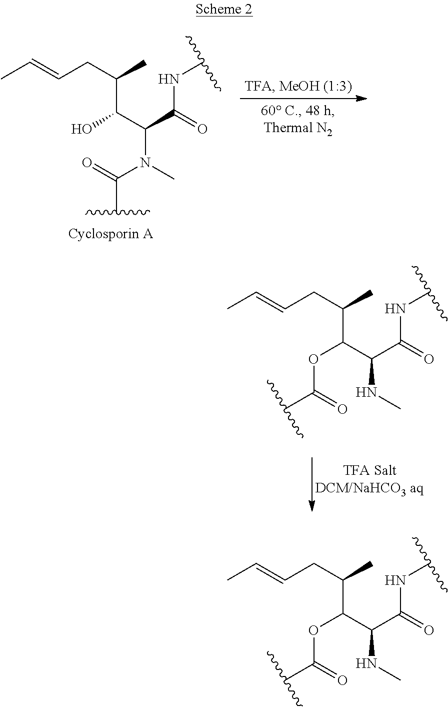 [SMILES: *NC(=O)[C@H]([C@H](O)[C@H](C)C/C=C/C)N(C)C(*)=O ; *NC(=O)[C@@H](NC)C(OC(*)=O)[C@H](C)C/C=C/C ; *NC(=O)[C@@H](NC)C(OC(*)=O)[C@H](C)C/C=C/C]

The excess of trifluoroacetic acid in step c) can be removed by stripping with diethyl ether under vacuum.

As it is possible to observe in the above indicated scheme 2, to the obtained salt of isocyclosporin A with trifluoroacetic acid DCM/NaHCO₃ can be added to remove the starting cyclosporin during the salification step (step d′).

After the salt of isocyclosporin A with trifluoroacetic acid is recovered, the method eventually comprises dissolving the salt of isocyclosporin A with trifluoroacetic acid obtained in step d) in a solution comprising an acid compound selected from citric acid and lactic acid and methanol.

In particular, the method according to the invention may comprise downstream of step d) the following steps:
e) dissolving an acid compound selected from citric acid and lactic acid in methanol;
f) dissolving said salt of isocyclosporin A with said trifluoroacetic acid in the solution obtained in step e) while stirring the resulting solution for a time ranging from 0.5 to 2 hours;
g) removing the methanol and said trifluoroacetic acid; and
h) recovering the salt of isocyclosporin A with said acid compound selected from citric acid and lactic acid.

Examples of preparation of salt of isocyclosporin A with an acid compound selected from citric acid and lactic acid are indicated in the experimental section (examples 3 and 4).

The Applicant has observed that with the optimal molar ratio between trifluoroacetic acid and methanol of 1:3 a conversion of 53% of the cyclosporin A in isocyclosporin A without having by-products is obtained, whereas with molar ratios between acid compound and methanol of 1:1 or 1:4 there is a lower conversion (30% or 20%) of the cyclosporin A in isocyclosporin A (see examples 1 and 2).

With a molar ratio of acid compound to methanol of 3:1 there is a higher conversion of cyclosporin A in isocyclosporin A (75%) but with a higher number of by-products.

The total yield of isocyclosporin A obtained by the above-described method is 80%.

According to the second embodiment of the present invention, the method for preparing a salt of isocyclosporin A by transesterification of cyclosporin A into a salt of isocyclosporin A comprises the steps of:
a) dissolving said cyclosporin A in anhydrous methanol and adding trifluoroacetic acid;
b) heating in a microwave oven the solution obtained according to step a);
c) removing said methanol and the excess of said trifluoroacetic acid; and
d) recovering the salt of isocyclosporin A with said trifluoroacetic acid.

In particular, in step b) of the method the solution obtained according to step a) is heated in the microwave at a temperature ranging from 55° C. to 65° C., preferably at the temperature of 60° C.

In particular, step b) is carried out for a time ranging from 10 to 20 hours, preferably of about 15 hours.

In a particularly preferred embodiment, the microwave heating according to step b) of the method is carried out at 60° C. for 15 hours.

In fact, under these conditions it is possible to obtain a yield of a salt of isocyclosporin A of 100%.

Scheme 3 below shows the reaction scheme according to the method of the invention wherein the reaction solution between the compound (trifluoroacetic acid) and methanol is heated in the microwave (M.W.):

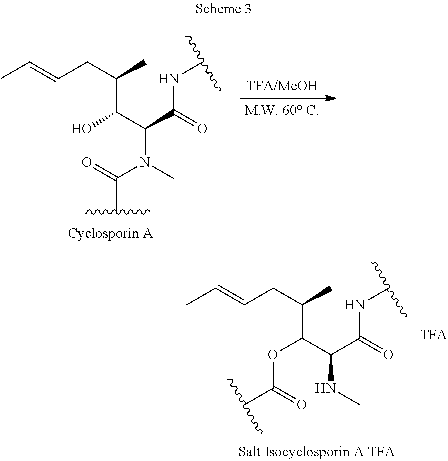 [SMILES: *NC(=O)[C@H]([C@H](O)[C@H](C)C/C=C/C)N(C)C(*)=O ; *NC(=O)[C@@H](NC)C(OC(*)=O)[C@H](C)C/C=C/C]

In step c) of the method according to the invention, the excess acid compound is removed by stripping with diethyl ether under vacuum.

After the salt of isocyclosporin A with the trifluoroacetic acid is recovered (step d), the method comprises eventually dissolving said salt of isocyclosporin A with said trifluoroacetic acid in a solution comprising an acid compound selected from citric acid and lactic acid and methanol.

In particular, the method according to the invention may comprise downstream of step d) the following steps:
e) dissolving an acid compound selected from citric acid and lactic acid in methanol;
f) dissolving said salt of isocyclosporin A with said trifluoroacetic acid in the solution obtained in step e) while stirring the resulting solution for a time ranging from 0.5 to 2 hours;
g) removing the methanol and said trifluoroacetic acid; and
h) recovering the salt of isocyclosporin A with said acid compound selected from citric acid and lactic acid.

Examples of preparation of salt of isocyclosporin A with an acid compound selected from citric acid and lactic acid are indicated in the experimental section (examples 3 and 4).

The method for preparing a salt of isocyclosporin A comprising microwave heating can be carried out by a continuous flow system which comprises one or more microwave reactors. In particular, the Applicant has observed that by combining the microwave heating with the continuous flow technique high yields of isocyclosporin A are obtained. Therefore, a second aspect of the second embodiment of the present invention refers to a continuous flow microwave system for preparing a salt of isocyclosporin A which comprises heating in the microwave oven the solution according to step a). In particular, the continuous flow microwave system for preparing a salt of isocyclosporin A comprises one or more dispensing units of starting reagents, represented by the solution obtained in step a) of the method according to the present invention, one or more microwave reactors and one or more product collectors.

In particular, in said system the starting reagents are transported from the dispensing units to microwave reactors by pumps, preferably HPLC pumps or syringe pumps.

The system may also comprise one or more coolers, and one or more back pressure regulators for monitoring the pressure.

Furthermore, there may also be sensors, as for example optical fiber sensors, for monitoring the reaction temperature.

In an embodiment, the system comprises a single dispensing unit of starting reagents, a single microwave reactor and a single product collector (as indicated in FIG. 4). In this reactor, there is also one pump, one cooler and one back pressure cooler.

Preferably, the continuous flow microwave system comprises more than one dispensing unit of starting reagents, more than one microwave reactor and more than one product collector. There may also be more than one pump, more than one cooler and more than one back pressure cooler.

More preferably, the continuous flow microwave system comprises multiple microwave reactors in parallel.

### DESCRIPTION OF FIGURES

FIG. 1 shows the liquid chromatography results of the salt of isocyclosporin A obtained by using a molar ratio of trifluoroacetic acid and methanol of 1:3. In FIG. 1 “IsoCsA” indicates isocyclosporin A and “CsA” indicates cyclosporin A.

FIG. 2 shows the liquid chromatography results of the salt of isocyclosporin A obtained by using a molar ratio of trifluoroacetic acid and methanol of 1:4 (see example 2). In FIG. 2 “IsoCsA” indicates isocyclosporin A and “CsA” indicates cyclosporin A.

FIG. 3 shows the liquid chromatography results of the salt of isocyclosporin A obtained by carrying out the heating of the reaction mixture in the microwave. In FIG. 3 “IsoCsA” indicates isocyclosporin A.

FIG. 4 is a graphical representation of the continuous flow microwave system according to the invention. In particular, the continuous flow microwave system shown in FIG. 4 comprises a dispensing unit (1), a microwave reactor (2) and a product collector (3). In FIG. 4 are also shown a pump (4) that conveys the starting reagents from the dispensing unit (1) to the microwave reactor (2), a cooler (5) and a back pressure regulator (6).

### EXAMPLES

### Example 1: Transesterification of Cyclosporin A into a Salt of Isocyclosporin A with Trifluoroacetic Acid-Molar Ratio TFA:Methanol of 1:3

The cyclosporin A (2.5 g, 2.08 mmoles) has been dissolved in anhydrous methanol (2.45 ml). Trifluoroacetic acid (TFA) (1.5 ml) has been added and the reaction has been stirred at reflux at 60° C. for 48 hours.

The solvent has been removed at reduced pressure and the excess of residual TFA has been removed by stripping with diethyl ether (2×15 ml) under vacuum.

The salt of Isocyclosporin A with dried TFA (1.33 g) looked like a white powder. A conversion of about 53% of the starting material and a quantitative yield of Iso-CsA have been obtained. In the final reaction no by-products were observed. The remaining starting material (cyclosporin A, CsA) has been removed during the step of salification by adding NAHCO₃.

As it can be observed from Table 1, the molar ratio between trifluoroacetic acid and methanol is 1:3.

TABLE 1

| | Molecular | | | | | |
|---|---|---|---|---|---|---|
| | weight | | Moles | Mass | Volume | Density |
| Reagent | gmol⁻¹ | Eq. | mmol | g | ml | gml⁻¹ |
| Cyclosporin A (CsA) | 1202.61 | / | 2.08 | 2.5 | / | / |
| AK Scientific Lot 22C4I30I | | | | | | |
| Trifluoroacetic acid (TFA) | 114.02 | / | 20 | / | 1.5 | 1.489 |

Product Yields: Quantitative

| | | | | | | |
|---|---|---|---|---|---|---|
| Salt isocyclosporin A with TFA | 1316.62 | / | 1.11 | 1.33 | / | / |
| (Iso-CsA · xTFA) | | | | | | |

MeOH(3x mmol of TFA) = 60 mmol = 2.45 ml,
d = 0.79 g/ml

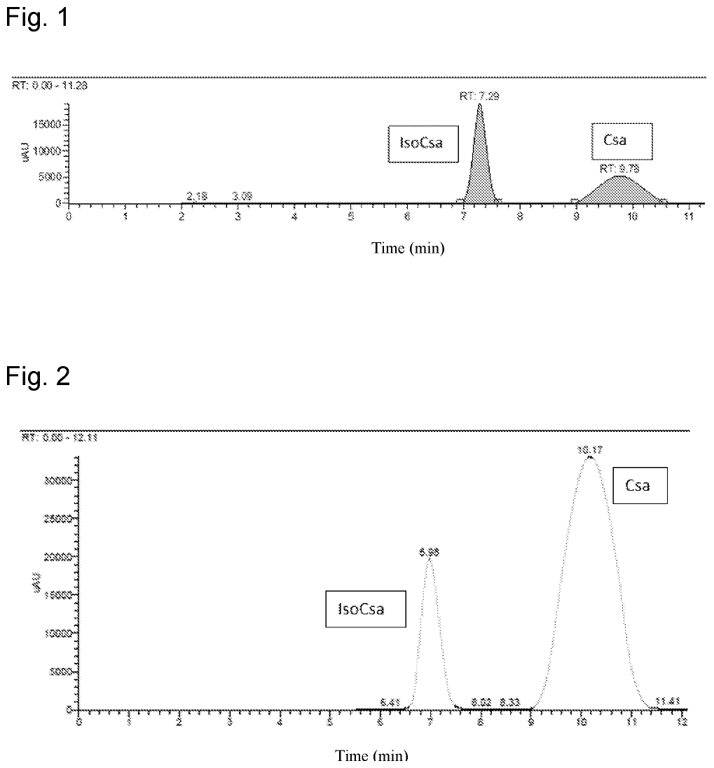

The product has been characterized through liquid chromatography (see FIG. 1).

### Example 2: Transesterification of Cyclosporin A into a Salt of Isocyclosporin A-Molar Ratio TFA:Methanol of 1:1, 1:4, 3:1

The Applicant has carried out other experiments changing the molar ratio between trifluoroacetic acid and methanol according to what is indicated in Table 2.

TABLE 2
Cyclosporin A (CsA) 2.5 gr (2.08 mmol) in salt Isocyclosporin A TFA

| | Molar | | | % | Volume | |
|---|---|---|---|---|---|---|
| | Ratio | | Mmol(TFA) | Conversion | MeOH | % Yield |
| Reagents | (TFA/MeOH) | Time(h) | mmol | (CsA) | ml | (IsoCsA) |
| a) Trifluoroacetic | 1:1 | >48 | 20 | 30 | 2.45 | 50 |
| acid | | | | | | |
| (TFA:MeOH) | | | | | | |
| b) Trifluoroacetic | 1:4 | >48 | 20 | 20 | 9.8 | 35 |
| acid | | | | | | |
| (TFA:MeOH) | | | | | | |
| c) Trifluoroacetic | 3:1 | 48 | 60 | 75 | 0.85 | / |
| acid | | | | | | (by-products) |
| (TFA:MeOH) | | | | | | |

As it can be observed from Table 2, with molar ratios between trifluoroacetic acid compound and methanol of 1:1 or 1:4 there is a lower conversion (30% or 20%) of cyclosporin A in isocyclosporin A. On the other hand, with a molar ratio between acid compound and methanol of 3:1 there is a greater conversion of cyclosporin A in isocyclosporin A (75%) but with a higher number of by-products.

The product has been characterized through liquid chromatography (see FIG. 2).

### Example 3: Preparation of a Salt of Isocyclosporin A Salt with Citric Acid

The salt of isocyclosporin A with trifluoroacetic acid (1 mmol) has been dissolved in a solution of MeOH with citric acid (1 mmol). The solution has been kept for 1 h under stirring, thereafter the solvent has been removed at reduced pressure and the excess of residual trifluoroacetic acid has been removed by stripping with diethyl ether (2×15 ml) under vacuum. 90% yield, of a salt of isocyclosporin A salt with citric acid.

### Example 4: Preparation of a Salt of Isocyclosporin A Salt with Lactic Acid

The salt of isocyclosporin A with trifluoroacetic acid (1 mmol) has been dissolved in a solution of MeOH with lactic acid (1 mmol). The solution has been kept for 1 h under stirring, thereafter the solvent has been reduced at reduced pressure and the excess of residual trifluoroacetic acid has been removed by stripping with diethyl ether (2×15 ml) under vacuum. 91% yield, of a salt of isocyclosporin A salt with lactic acid.

### Example 5: Transesterification of Cyclosporin A into a Salt of Isocyclosporin A-Microwave Heating

The cyclosporin A (2.5 g, 2.08 mmoles) has been dissolved in anhydrous methanol, then trifluoroacetic acid has been added (5 ml) and the reaction vial has been heated in the microwave at 60° C. for 15 hours, by using the Biotage MW reactor.

The solvent has been reduced at reduced pressure and the excess of residual trifluoroacetic acid has been removed by stripping with diethyl ether (2×15 ml) under vacuum.

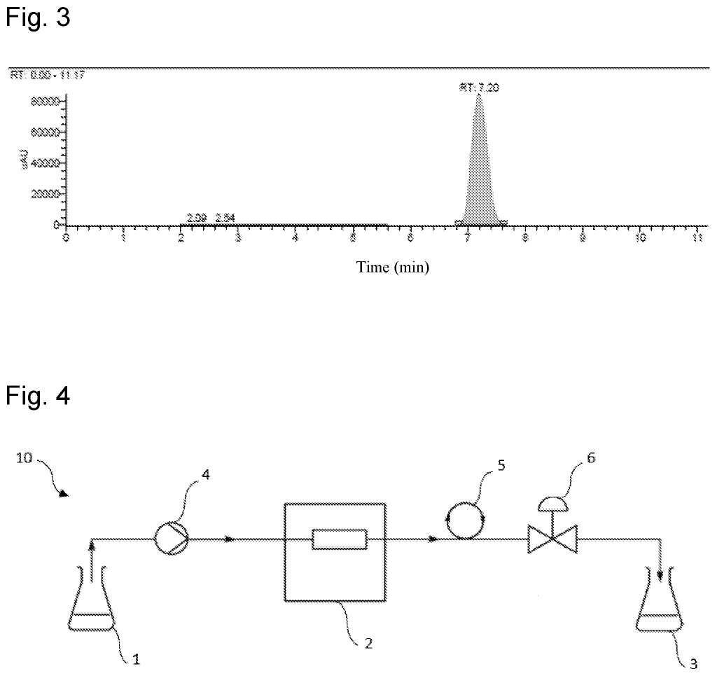

The salt of Isocyclosporin A with dried TFA (3.386 g) looked like a white powder. The product has been characterized through liquid chromatography (see FIG. 3).

### Example 6: Transesterification of Cyclosporin A into a Salt of Isocyclosporin A-Temperature of 55° C.

The preparation of example 5 has been repeated by varying the reaction temperature. In particular, the cyclosporin A (2.5 g, 2.08 mmoles) has been dissolved in anhydrous methanol, then trifluoroacetic acid has been added (5 ml) and the reaction vial has been heated in the microwave at 55° C. for 15 hours, by using the Biotage MW reactor. The solvent has been reduced at reduced pressure and the excess of residual trifluoroacetic acid has been removed by stripping with diethyl ether (2×15 ml) under vacuum.

3.008 g of salt of Isocyclosporin A with dried TFA have been obtained.

### Example 7: Transesterification of Cyclosporin A into a Salt of Isocyclosporin A-Reaction Time 10 Hours

The preparation of example 5 has been repeated by varying the reaction time.

In particular, cyclosporin A (2.5 g, 2.08 mmoles) has been dissolved in anhydrous methanol, then trifluoroacetic acid has been added (5 ml) and the reaction vial has been heated in the microwave at 60° C. for 10 hours, by using the Biotage MW reactor.

The solvent has been reduced at reduced pressure and the excess of residual trifluoroacetic acid has been removed by stripping with diethyl ether (2×15 ml) under vacuum.

After 10 hours a quantity of product equal to 80% of the yield observed at 15 hours was obtained.

### Example 8: Transesterification of Cyclosporin A into a Salt of Isocyclosporin A-Reaction Time 20 Hours

The preparation of example 5 has been repeated by varying the reaction time.

In particular, cyclosporin A (2.5 g, 2.08 mmoles) has been dissolved in anhydrous methanol, then trifluoroacetic acid has been added (5 ml) and the reaction vial has been heated in the microwave at 60° C. for 20 hours, by using the Biotage MW reactor.

The solvent has been reduced at reduced pressure and the excess of residual trifluoroacetic acid has been removed by stripping with diethyl ether (2×15 ml) under vacuum.

After 20 hours an increase of 2% with respect to the yield observed at 15 hours was observed.

1.-10. (canceled)

11. A method for preparing a salt of isocyclosporin A by transesterification of cyclosporin A into a salt of isocyclosporin A, the method comprising the steps of:
(a) dissolving the cyclosporin A in anhydrous methanol and adding trifluoroacetic acid, thereby obtaining a solution of dissolved cyclosporin A;
(b) heating the solution obtained in step (a) by microwave radiation;
(c) removing a liquid comprising the anhydrous methanol and the trifluoroacetic acid, thereby obtaining a residue; and
(d) recovering the salt of isocyclosporin A with the trifluoroacetic acid from the residue.

12. The method of claim 11, wherein step (b) is carried out at a temperature of about 55° C. to about 65° C.

13. The method of claim 11, wherein step (b) is carried out at time ranging from about 10 to about 20 hours.

14. The method of claim 11, wherein step (b) is carried out at time of about 15 hours.

15. The method of claim 11, wherein the trifluoroacetic acid and the anhydrous methanol in the solution obtained in step (a) is at a molar ratio of 1:3.

16. The method of claim 11, wherein the liquid comprising the trifluoroacetic acid is removed by stripping with diethyl ether under a vacuum.

17. The method of claim 11, wherein the solution is heated at a temperature of about 60° C.

18. The method of claim 11, wherein the solution is heated at a temperature of about 60° C. for a time of about 15 hours.

19. The method of claim 11, wherein recovering the salt of isocyclosporin A with the trifluoroacetic acid comprises contacting the residue with DCM/NaHCO₃ to remove cyclosporin A.

20. The method of claim 11, further comprising, after step (d), the following steps of:
(e) dissolving an acid selected from citric acid or lactic acid in methanol, thereby generating a dissolved acid solution;
(f) dissolving the salt of isocyclosporin A with the trifluoroacetic acid in the dissolved acid solution obtained in step (e) while stirring for a time ranging from 0.5 to 2 hours; and
(g) removing the methanol and the trifluoroacetic acid to obtain the salt of isocyclosporin A with citric acid or lactic acid.

21. The method of claim 11, wherein step (a) comprises dissolving about 2 mmol of cyclosporin A in about 60 mmol of methanol.

22. A composition comprising the salt of isocyclosporin A with the acid selected from citric acid and lactic acid prepared by the method of claim 20.

23. A continuous flow microwave system configured to carry out the method of claim 11.

24. The continuous flow microwave system of claim 23, comprising one or more dispensing units of starting reagents, one or more microwave reactors, and one or more product collectors.

25. The continuous flow microwave system of claim 24, further comprising one or more pumps for conveying the starting reagents from the one or more dispensing units to the one or more microwave reactors.

26. The continuous flow microwave system of claim 25, wherein the one or more pumps comprise an HPLC pump or a syringe pump.

27. The continuous flow microwave system of claim 24, further comprising one or more coolers, and one or more back pressure regulators.

28. The continuous flow microwave system of claim 24, wherein microwave reactors of the one or more microwave reactors are in parallel.

29. A continuous flow microwave system configured to carry out the method of claim 20.
